(19) United States
(12) Patent Application Publication
(10) Pub. No.: US 2024/0285400 A1
(43) Pub. Date: Aug. 29, 2024

(54) PROSTHETIC HEART VALVE

(71) Applicant: EDWARDS LIFESCIENCES CORPORATION, Irvine, CA (US)

(72) Inventors: Eran Grosu, Kfar-Saba (IL); Nikolai Gurovich, Hadera (IL); Michael Bukin, Pardes Hanna (IL); Anatoly Dvorsky, Haifa (IL); Noam Nir, Pardes-Hanna (IL); Joseph Mordechai Leichner, Netanya (IL); Ziv Yohanan, Kfar Hahoresh (IL)

(21) Appl. No.: 18/656,741

(22) Filed: May 7, 2024

Related U.S. Application Data
(63) Continuation of application No. PCT/US2022/049436, filed on Nov. 9, 2022.
(60) Provisional application No. 63/280,020, filed on Nov. 16, 2021.

(51) Int. Cl. A61F 2/24; A61F 2/00

(52) U.S. Cl. CPC A61F 2/2418; A61F 2/243; A61F 2002/0081; A61F 2230/0017; A61F 2250/001

(57) ABSTRACT

Examples of prosthetic heart valves and frames for use in prosthetic heart valve are disclosed herein. The prosthetic heart valves comprise frames that are movable between a radially compressed configuration and a radially-expanded configuration and comprise a row of inlet cells, a row of outlet cells, and a row of intermediate cells. The frames of the prosthetic heart valves include actuation members and locking mechanisms configured to secure the frame in one or more radially-expanded configurations. In some examples, the actuation members and locking members are integrally formed with the frame. The frames of the prosthetic heart valves can be coupled to one or more shafts of a delivery apparatus via a threaded connection or a non-threaded connection.

### CROSS-REFERENCE TO RELATED APPLICATIONS

The present application is a continuation of PCT Application No. PCT/US2022/049436, filed Nov. 9, 2022, which claims the benefit of U.S. Provisional Application No. 63/280,020, filed Nov. 16, 2021, both of which are incorporated by reference herein.

### FIELD

The present disclosure relates to implantable expandable prosthetic heart valves and frame structures for use with expandable prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves are designed to withstand various pressures from the native anatomy of the patient in which they are implanted, and to provide adequate support and protection for soft components such as the leaflets of the valvular structure of the prosthetic heart valve. Additionally, stability of the individual cells of the frame may be a concern, as compressive forces from the native annulus of a patient can cause a frame with insufficiently stable cells to buckle or collapse. Furthermore, frames with large gaps between cells may inadequately support a sealing member, which is disposed around the outside of the frame and seals the prosthetic heart valve against the implantation site and prevents or reduces undesirable blood flow around the exterior of the prosthetic heart valve.

Accordingly, there is a need for improved prosthetic heart valve frames with superior mechanical stability and improved support for the attached soft components of a prosthetic heart valve.

### SUMMARY

Disclosed herein are prosthetic heart valve frames having an increased number of cells and greater mechanical stability. The disclosed prosthetic heart valve frames can offer superior resistance to the forces of the native anatomy of the patient by reducing spaces along the inflow and outflow end portions of the prosthetic heart valve through the use of additional inflow and outflow cells. Additionally, the prosthetic heart valve frames disclosed herein can improve the mechanical stability of the prosthetic heart valve by introducing additional vertical struts to define a greater number of smaller, more stable intermediate cells between the inflow and outflow end portions. As such, the prosthetic heart valve frames disclosed herein can, among other things, overcome the challenges of providing adequate support and protection to the soft components of a prosthetic heart valve's valvular structure and sufficient mechanical stability against the forces exerted on the prosthetic heart valve by the patient's anatomy.

Certain examples concern a prosthetic heart valve comprising a frame and a valve structure coupled to the frame. In addition to these components, a prosthetic heart valve can further comprise one or more of the components disclosed herein.

In some examples, a prosthetic heart valve can comprise a sealing member configured to reduce paravalvular leakage.

In some examples, a prosthetic heart valve can include a plurality of inflow cells, a plurality of outflow cells, and a plurality of intermediate cells disposed between the inflow cells and the outflow cells.

In some examples, the prosthetic heart valve can include a plurality of actuated cells, and a plurality of non-actuated cells.

In some examples, the number of inflow cells and the number of outflow cells can be greater than the number of intermediate cells.

In some examples, the inflow cells and the outflow cells are circumferentially aligned and arranged in one or more pairs of inflow cells and outflow cells.

In some examples, the prosthetic heart valve can comprise a plurality of axially extending struts interconnecting the one or more pairs of inflow and outflow cells.

In some examples, the prosthetic heart valve can comprise an actuation member extending through a first axially extending strut of the plurality of axially extending struts and an inflow cell and an outflow cell of a selected pair of inflow and outflow cells from the pairs of inflow and outflow cells.

In some examples, the prosthetic heart valve comprises two or more actuation members.

In some examples, the inflow and outflow cells are diamond shaped and the intermediate cells are hexagonal.

In some examples, the one or more pairs of inflow cells and outflow cells comprise a first set of pairs and a second set of pairs, with each pair of inflow cells and outflow cells of the first set of pairs is located circumferentially between two pairs of inflow cells and outflow cells of the second set of pairs.

In some examples, the outflow cells of the second set of pairs define first apices and the outflow cells of the first set of pairs define second apices.

In some examples, a first distance between the first apices and an axial mid-section of the frame is less than a second distance between the second apices and the axial mid-section of the frame.

In some examples, a first distance between the first apices and an axial mid-section of the frame is greater than a second distance between the second apices and the axial mid-section of the frame.

In some examples, the frame of the prosthetic heart valve comprises a circumferentially extending row of hexagonal cells, each hexagonal cell defined by two adjacent axially extending struts, a first strut of an inflow cell, a second strut of another, adjacent inflow cell, a third strut of an outflow cell, and a fourth strut of another, adjacent outflow cell.

Certain examples concern a prosthetic heart valve assembly, comprising a radially expandable annular frame and a valvular structure disposed within the frame and configured to regulate the flow of blood through the frame from the inflow end to the outflow end. The radially expandable annular frame comprises a plurality of interconnected struts that form an inflow row of cells defining an inflow end of the frame and a plurality of interconnected struts that form an outflow row of cells defining an outflow end of the frame and circumferentially aligned with corresponding cells of the inflow row. The radially expandable annular frame also comprises a plurality of axially extending struts interconnecting pairs of corresponding inflow and outflow cells, and at least one actuation member extending through one of the axially extending struts and an inflow cell and an outflow cell of a selected pair of inflow and outflow cells from the pairs of corresponding inflow and outflow cells, wherein the actuation member is configured to radially expand and compress the frame.

Certain examples concern a radially expandable frame comprising a row of inflow cells circumferentially arranged in an annular configuration at an inflow end portion of the frame and a row of outflow cells circumferentially arranged in an annular configuration at an outflow end portion of the frame, wherein each inflow cell is circumferentially aligned with an outflow cells to define pairs of circumferentially aligned inflow and outflow cells. The radially expandable frame also comprises a row of intermediate cells circumferentially arranged in an annular configuration and disposed between the row of inflow cells and the row of outflow cells and a plurality of axially extending vertical struts connecting pairs of inflow and outflow cells and forming sides of the intermediate cells. The radially expandable frame further comprises at least one actuation device extending from an inflow cell to an outflow cell of a selected pair of inflow and outflow cells, wherein the actuation device is configured to radially expand frame from a radially compressed state to a radially expanded state.

Certain examples concern a radially expandable frame, comprising a plurality of diamond-shaped outflow cells, a plurality of diamond-shaped inflow cells, and plurality of hexagonal-shaped intermediate cells disposed between the outflow cells and the inflow cells. The radially expandable frame also comprises at least one actuator extending from an inflow cell to an outflow cell and configured to radially expand and radially contract the radially expandable frame.

Certain examples concern a radially expandable frame, comprising an inflow row of cells defining an inflow end of the radially expandable frame and an outflow row of cells defining an outflow end of the radially expandable frame. The radially expandable frame also comprises a plurality of axially extending vertical struts connecting the inflow row of cells with the outflow row of cells to define a plurality of pairs of end cells comprising an inflow cell and a corresponding outflow cell. The radially expandable frame further comprises an intermediate row of cells disposed between the inflow row of cells and the outflow row of cells and defined by two vertical struts, two cells of the inflow row of cells, and two cells of the outflow row of cells. The radially expandable frame includes at least one actuator extending from the inflow cell of a pair of end cells to the corresponding outflow cell of the pair of end cells. Each inflow cell has an inflow apex at the inflow end of the radially expandable frame and an interior vertex located between the inflow apex and the corresponding outflow cell, and each outflow cell has an outflow apex at the outflow end of the radially expandable frame and an interior vertex located between the outflow apex and the corresponding inflow cell, such that the interior vertices of each pair of end cells face each other and the cells of the intermediate row of cells are larger than the cells of the outflow row of cells and the cells of the inflow row of cells.

Certain examples concern a radially expandable frame, comprising a plurality of frame sections. The plurality of frame sections comprises a plurality of circumferentially-extending interconnecting angled struts and a plurality of axially-extending vertical struts. The plurality of frame sections further comprises a diamond-shaped first inflow cell defined by four interconnected angled struts and having a first inflow apex, a diamond-shaped first outflow cell defined by four interconnected angled struts and having a first outflow apex, and two hexagonal-shaped intermediate cells defined by two vertical struts and four angled struts. The first inflow cell of each frame section is circumferentially aligned with the first outflow cell of each frame section and the hexagonal shaped intermediate cells of each frame section are axially aligned with each other, and the axially-extending vertical struts extend from the first inflow cell of each frame section to the first outflow cell of each frame section. The radially expandable frame also comprises a plurality of second inflow cells each having a second inflow apex and disposed between the first inflow cells of two frame sections, a plurality of second outflow cells, each having a second outflow apex and disposed between the first outflow cells of two frame sections, and at least one actuation member extending from a first inflow cell of a frame section to the corresponding outflow frame cell of that frame section.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 depicts a prosthetic heart valve according to one example disclosed herein, shown in the radially expanded configuration.

FIG. 2 depicts the frame of the prosthetic heart valve of FIG. 1.

FIG. 3 depicts a frame of a prosthetic heart valve according to one example disclosed herein, shown in the radially contracted configuration.

FIG. 4 depicts a cutaway view of a portion of the prosthetic heart valve frame of FIG. 3.

FIG. 5 depicts a segment of a frame according to one example herein, incorporating a locking mechanism.

FIG. 6 depicts the locking mechanism of FIG. 5.

FIG. 7 depicts a cutaway view of a portion of a prosthetic heart valve frame according to one example disclosed herein, having axially-extending bores to accommodate actuators.

FIG. 8 depicts an exemplary delivery device suitable for use with the prosthetic heart valves disclosed herein.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,”“an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Introduction to the Disclosed Technology

Disclosed herein are various examples of prosthetic heart valve frames having crossing frame members. The prosthetic heart valve frames disclosed herein can have a first row of cells disposed along the inflow end of the frame, a second row of cells disposed along the outflow end of the frame, and a third row of cells disposed between the first row of cells and the second row of cells. Cells of the first row can be circumferentially aligned with corresponding cells of the second row to form cell pairs, which may be connected by vertical struts extending therebetween. The prosthetic heart valves having frames disclosed herein can be inserted into the vasculature of a patient while in a radially compressed state and then expanded to a desired diameter by an expansion mechanism, such as an actuator, when in the desired implantation location (for example, within one of the native heart valves). The frames can also be locked in the desired state of radial expansion and prevented from further radial expansion or compression by means of a locking mechanism.

A prosthetic heart valve frame can face several technical challenges during and following the implantation procedure. The prosthetic heart valve frame must be sufficiently flexible to expand from the compressed state to the expanded state without failure, but must also have sufficient structural rigidity to withstand radially compressive forces, such as from the surrounding tissues of the patient's native annulus, without radially collapsing. In addition to these structural requirements, the frame must also have adequate features for attaching and supporting soft components of the prosthetic valve, such as prosthetic valve leaflets, an inner skirt, and/or an external skirt (also referred to as a “sealing member”).

Both challenges can be overcome by the addition of additional diagonal struts at the inflow and outflow ends of the frame. These additional struts can increase the number of frame cells in the cell rows at the inflow and outflow ends (that is, the inflow row of cells and the outflow row of cells) of the prosthetic heart valve frame. A frame with additional cells in the inflow row of cells and the outflow row of cells can have improved strength and rigidity, especially when resisting compressive forces such as might be exerted on the frame by the native annulus of the patient. Furthermore, the additional cells in the inflow and outflow row of cells do not need to include an actuator and may therefore be more easily used to attach various soft components of the prosthetic valve, such as valve leaflets, an inner skirt and/or an outer skirt.

This disclosure describes prosthetic heart valve frames that improve the strength and stability of the prosthetic heart valves in which they are used, and facilitate the attachment of soft components such as skirts and leaflets. These frames comprise additional diagonal and/or angular struts positioned at the inflow and outflow ends of the frame, which form additional frame cells at the inflow and outflow ends of the frame.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

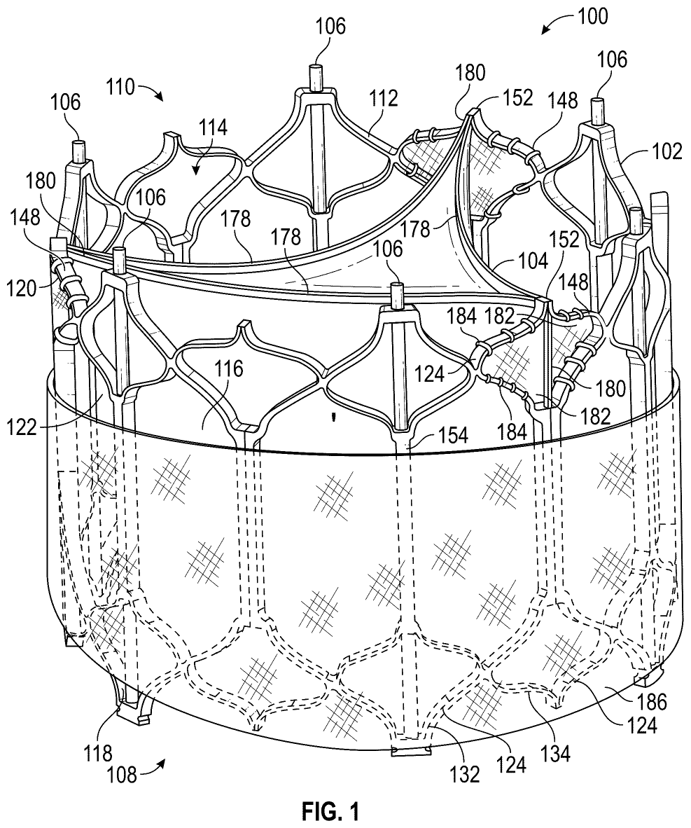

FIG. 1 shows an exemplary prosthetic valve 100, according to one example. Any of the prosthetic valves disclosed herein are adapted to be implanted in the native aortic annulus, although in some examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries, and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For instance, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

### The Disclosed Technology and Examples

FIG. 1 depicts one example of a prosthetic heart valve, which can be radially compressed for delivery through a patient's vasculature and radially expanded to a functional size at a desired implantation location within the patient' body (for example, the native aortic valve). The prosthetic heart valve 100 (also referred to herein as “the prosthetic valve 100”) comprises a frame 102 and a valvular structure 104.

The frame 102 (which can also be referred to as “a stent” or “a support structure”) can be configured to support the valvular structure 104 and for securing the prosthetic heart valve 100 within a native heart valve and/or within another support structure (for example, an anchoring frame (such as a coil) and/or a previously implanted prosthetic heart valve (that is, in a valve-in-valve procedure). The frame 102 can further comprise one or more actuators 106 configured to radially expand or radially compress the frame 102, as further described in detail below.

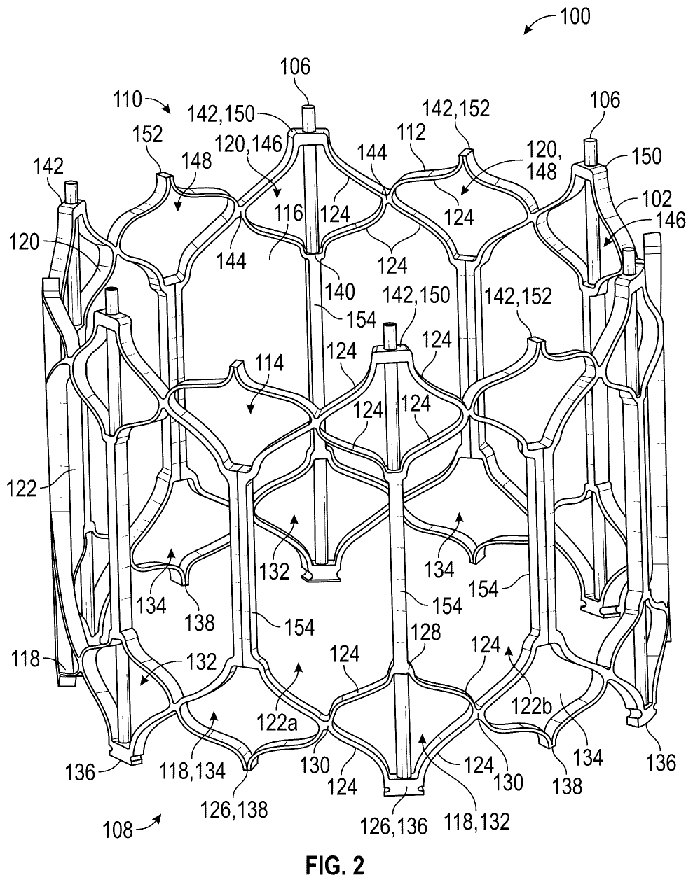

With continued reference to FIGS. 1-2, the frame 102 of the prosthetic heart valve 100 has a first end 108 and a second end 110. In the depicted orientation, the first end 108 of the frame 102 is an inflow end and the second end 110 of the frame 102 is an outflow end. In some examples, the first end 108 of the frame 102 can be the outflow end and the second end 110 of the frame 102 can be the inflow end.

The frame 102 can comprise a plurality of interconnected struts 112. In some examples, the struts 112 define a plurality of cells. In some examples, such as the example illustrated in FIGS. 1-2, the plurality of cells can further comprise a plurality of diamond-shaped cells 114 and a plurality of hexagonal cells 116. While FIGS. 1 and 2 show a prosthetic heart valve frame 102 comprising only hexagonal and diamond shaped cells, it is to be understood that other cellular geometries may also be included in prosthetic heart valve frame 102.

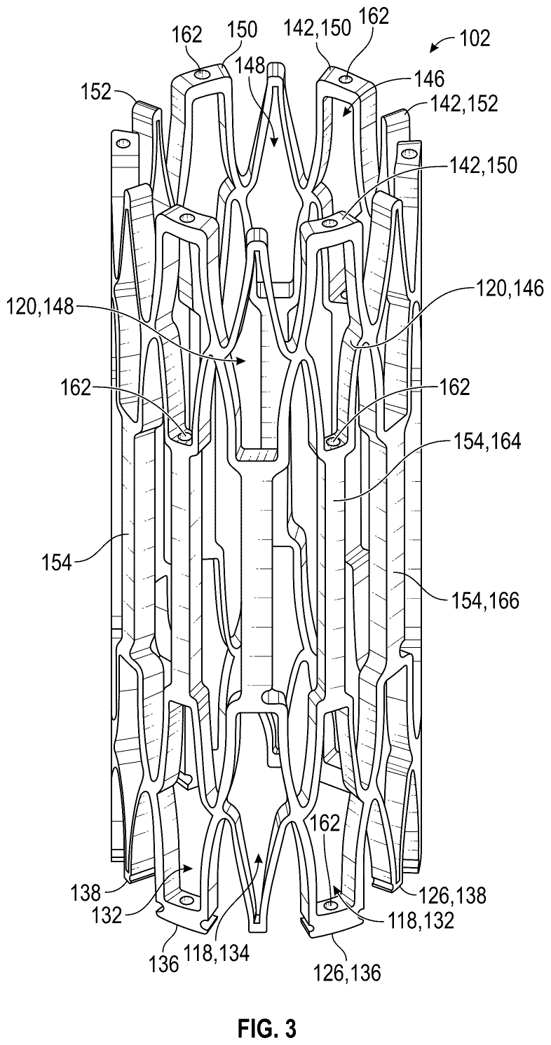

The frame 102 of the prosthetic heart valve 100 can be configured to radially expand from a radially compressed state to a radially expanded state. The frame 102 in the radially expanded state can be seen, for example, in FIGS. 1 and 2, and the frame 102 in the radially compressed state can be seen, for example, in FIG. 3. A prosthetic heart valve can be delivered through the vasculature of a patient to the desired implantation site (for example, a native heart valve of a patient) while in the radially compressed state. Once at the desired implantation site, the prosthetic heart valve can be radially expanded to the desired operational size, for example, through the use of one or more actuators, discussed in greater detail below.

In some examples, the plurality of cells can be arranged in circumferentially extending rows. For example, in the example shown in FIGS. 1-2, the struts 112 can define a row of inflow cells 118 defining the first end (or inflow end) 108 of the frame 102, a row of outflow cells 120 defining the second end (or outflow end) 110 of the frame 102, and a row of intermediate cells 122 extending from the row of inflow cells 118 to the row of outflow cells 120. In some examples, such as that illustrated in FIGS. 1-2, each inflow cell 118 and each outflow cell 120 is a diamond-shaped cell 114 and each intermediate cell 122 is a hexagonal cell 116. However, it is to be understood that each of the circumferential rows of cells (that is, inflow cells 118, outflow cells 120, and intermediate cells 122) can comprise cells of other geometries, or even a mix of cell geometries.

In some examples, such as that shown in FIGS. 1 and 2, the row of inflow cells 118 may comprise 12 cells such as 12 diamond-shaped cells 114, the row of outflow cells 120 may comprise 12 cells such as 12 diamond-shaped cells 114, and the row of intermediate cells 122 may comprise 12 cells such as 12 hexagonal cells 116. However, it is to be understood that in some examples, the frame 102 may include a lesser number of inflow cells, outflow cells, and/or intermediate cells, such as 6, 7, 8, 9, 10, or 11, or a greater number of inflow cells, outflow cells, and/or intermediate cells such as 13, 14, 15, or 16. Additionally, it is to be understood that, while FIGS. 1 and 2 show an example in which the frame 102 comprises an equal number of inflow cells, outflow cells, and intermediate cells, in some examples, the number of inflow cells, outflow cells, and intermediate cells may be different.

The frame 102 of the prosthetic heart valve 100 can comprise a row of inflow cells 118 disposed along and defining an inflow end of the prosthetic heart valve frame 102. As best illustrated in FIG. 2, the inflow cells 118 can be defined by a plurality of diagonal struts 124. The diagonal struts 124 can intersect to define a diamond-shaped inflow cell 118, having a distal inflow cell vertex 126, a proximal inflow cell vertex 128, and two lateral inflow cell vertices 130. The distal inflow cell vertex 126 of the inflow cell 118 can define an inflow apex of the frame 102, and the proximal inflow cell vertex 128 can define an interior vertex of the inflow cell 118. Each inflow cell 118 can be connected with an adjacent inflow cell 118 at each lateral inflow cell vertex 130 to form an annular ring of inflow cells 118 that defines the inflow end 108 of the frame 102.

In the illustrated example, two of the diagonal struts 124 defining each inflow cell 118 can also partially define an intermediate cell 122, such as adjacent intermediate cells 122a and 122b shown in FIG. 2.

With continued reference to FIG. 2, the inflow cells 118 may include a first set of inflow cells 132 and a second set of inflow cells 134. In some examples, the first set of inflow cells 132 and the second set of inflow cells 134 can comprise an equal number of inflow cells 118. In such examples, each inflow cell 132 can be located between two inflow cells 134, and each cell 134 can be located between two inflow cells 132 (that is, the inflow cells 132 alternate with the inflow cells 134 around the circumference of the frame). It is to be understood, however, that there may be a different number of inflow cells 132 than inflow cells 134. In such examples, it is to be further understood that two adjacent inflow cells 132 may have no inflow cell 134 disposed therebetween, and/or two adjacent inflow cells 134 may have no inflow cell 132 disposed therebetween.

The first set of inflow cells 132 can comprise a first set of inflow apices 136 and the second set of inflow cells 134 can comprise a second set of inflow apices 138. As illustrated in FIG. 2, the axial distance between each inflow apex 136 and an axial midpoint of the frame 102 can, in some examples, be greater than the axial distance between each inflow apex 138 and the axial midpoint of the frame. For example, the inflow apices 136 define the inflow-most locations on the frame and the inflow apices 138 are offset from the inflow apices 136 in an upstream location toward the axial midpoint of the frame. It is to be understood, however, that in some examples, the first set of inflow apices 136 and the second set of inflow apices 138 can be positioned an equal distance from the axial midpoint of the frame 102. In yet other examples, the axial distance between each of inflow apex 136 and the axial midpoint of the frame 102 can be less than the axial distance from each apex apices 138 to the axial midpoint of the frame 102.

The frame 102 of the prosthetic heart valve 100 can comprise a row of outflow cells 120 disposed along and defining an outflow end 110 of the prosthetic heart valve frame 102. As best illustrated in FIG. 2, the outflow cells can be defined by a plurality of diagonal struts 124. The diagonal struts 124 can intersect to define a diamond-shaped outflow cell 120, having a distal outflow cell vertex 140, a proximal outflow cell vertex 142, and two lateral outflow cell vertices 144. The proximal outflow cell vertex 142 of the outflow cell 120 can define an outflow apex of the frame, and the distal outflow cell vertex 140 can define an interior vertex of the outflow cell. Each outflow cell 120 can be connected with an adjacent outflow cell 120 at each lateral outflow cell vertex 144 to form an annular ring of outflow cells that defines the outflow end 110 of the frame 102.

In the illustrated example, two of the diagonal struts 124 defining each outflow cell 120 can also partially define an intermediate cell 122, such as adjacent intermediate cells 122a and 122b shown in FIG. 2.

With continued reference to FIG. 2, the outflow cells 120 may include a first set of outflow cells 146 and a second set of outflow cells 148. In some examples, the first set of outflow cells 146 and the second set of outflow cells 148 can comprise an equal number of outflow cells 120. In such examples, each inflow cell 132 can be located between two inflow cells 134, and each cell 134 can be located between two inflow cells 132 (that is, the inflow cells 132 alternate with the inflow cells 134 around the circumference of the frame). In such examples, each outflow cell 146 can be located between two outflow cells 148, and each outflow cell 148 can be located between two outflow cells 146 (that is, the outflow cells 146 alternate with the outflow cells 148 around the circumference of the frame). It is to be understood, however, that there may be a different number of outflow cells 146 than outflow cells 148. In such examples, it is to be further understood that two adjacent outflow cells 146 may have no outflow cell 148 disposed therebetween, and/or two adjacent outflow cells 148 may have no outflow cell 146 disposed therebetween.

The first set of outflow cells 146 can comprise a set of first outflow apices 150 and the second set of outflow cells 148 can comprise a set of second outflow apices 152. As illustrated in FIGS. 1-2, the axial distance between each outflow apex 150 and an axial midpoint of the frame 102 can, in some examples, be greater than the axial distance between each outflow apex 152 and the axial midpoint of the frame. For example, the outflow apices 150 define the outflow-most locations on the frame and the outflow apices 152 are offset from the outflow apices 150 in a downstream location toward the axial midpoint of the frame. It is to be understood, however, that in some examples, the set of first outflow apices 150 and the set of second outflow apices 152 can be positioned an equal distance from the axial midpoint of the frame 102. In yet other examples, the distance between each outflow apex 150 and the axial midpoint of the frame 102 can be less than the distance from each outflow apex 152 and the axial midpoint of the frame 102.

In some examples, such as the examples illustrated in FIGS. 1-2, inflow cells 118 can be circumferentially aligned with corresponding outflow cells 120 to form pairs of inflow and outflow cells. Pairs of inflow and outflow cells may be connected by axially-extending vertical struts 154 that extend from the interior inflow cell vertex 128 of an inflow cell 118 to the interior outflow cell vertex 140 of a corresponding outflow cell 120. In the example shown in FIGS. 1 and 2, each inflow cell 118 forms a cell pair with a corresponding outflow cell 120, and each outflow cell 120 forms a cell pair with a corresponding inflow cell 118, (that is, the number of inflow cells is equal to the number of outflow cells, and each inflow cells is circumferentially aligned with a corresponding outflow cell to form a pair of inflow and outflow cells). However, it is to be understood that in some examples, there may be a greater number of inflow cells than outflow cells or a greater number of outflow cells than inflow cells, and that some inflow cells and some outflow cells may therefore not be included in pairs of inflow cells and outflow cells.

With continued reference to FIG. 2, the circumferentially aligned pairs of inflow and outflow cells can comprise a first set of aligned pairs and a second set of aligned pairs. The first set of aligned pairs can comprise circumferentially aligned cells from the first set of inflow cells 132 having first inflow apices 136 and the first set of outflow cells 146 having first outflow apices 150. The second set of aligned pairs can comprise circumferentially aligned cells from the second set of inflow cells 134 having second inflow apices 138 and the second set of outflow cells 148 having second outflow apices 152. In some examples, such as that illustrated in FIGS. 1-2, one or more actuators 106 can extend from the inflow apices 136 to the first outflow apices 150 of the first set of aligned pairs, while no actuators 106 extend from the second inflow apices 138 to the second outflow apices 152 of the second set of aligned pairs. While FIGS. 1-2 show an example in which each cell pair of the first set of cell pairs (that is, six cell pairs in the illustrated example) has an actuator 106, it is to be understood that fewer than all cell pairs of the first set of cell pairs can include an actuator. For example, 1, 2, 3, 4, or 5 cell pairs can have an actuator 106, and the remaining cell pairs of the first set of cell pairs can omit actuator 106.

The frame 102 of the prosthetic heart valve 100 can comprise a row of intermediate cells 122 disposed between and extending from the row of inflow cells 118 and the row of outflow cells 120. As best illustrated in FIG. 2, each intermediate cell can be defined by a combination of diagonal struts 124 and axially-extending vertical struts 154. For example, as illustrated in FIG. 2, each intermediate cell 122 can be defined by two intersecting diagonal struts 124 (from two adjacent inflow cells 118), two intersecting diagonal struts 124 (from two adjacent outflow cells 120), and two axially-extending vertical struts 154. In such an example, the intermediate cells 122 can have a hexagonal geometry. While FIG. 2 shows intermediate cells defined by four diagonal struts and two axially extending vertical struts, it is to be understood that in some examples, a different number of horizontal or axially extending vertical struts, or a different type of strut could define the intermediate cells 122, and the intermediate cells 122 could accordingly have a different geometry.

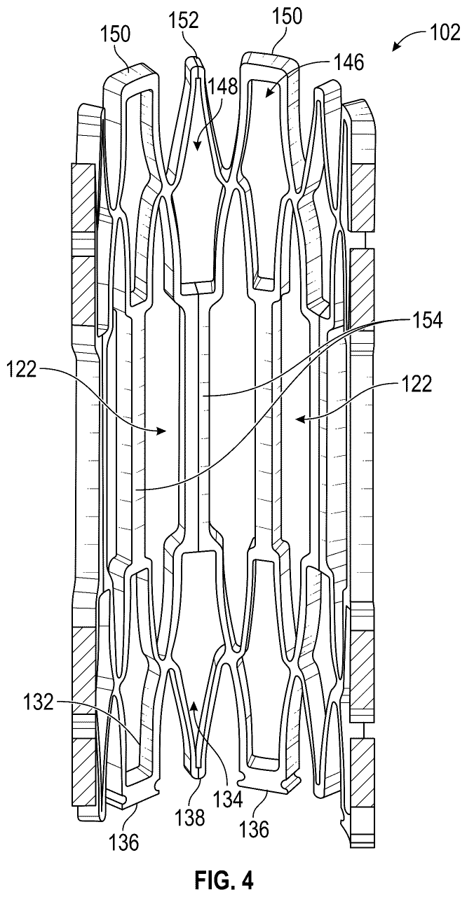

In some examples, such as those shown in FIGS. 1-4, the intermediate cells 122 can be immediately adjacent to other intermediate cells 122 along the axially extending vertical struts 154 (that is, two adjacent intermediate cells 122 can share an axially-extending vertical strut 154). In such examples, the inflow cells 118 and/or outflow cells 120 can be circumferentially offset from the intermediate cells 122. In certain examples, the axially extending vertical struts 154 can extend from the interior vertex 128 of the inflow cell 118 of a pair of inflow and outflow cells to the interior vertex 140 of the corresponding outflow cell 120 of the pair of inflow and outflow cells. Adjacent inflow cells 118 may, in such examples, connect only at lateral inflow cell vertices 130 and adjacent outflow cells 120 can connect only at lateral outflow cell vertices 144.

As noted above, the frame 102 of the prosthetic heart valve 100 can further include one or more actuators 106 configured to axially extend and foreshorten the frame 102 (and thereby to radially expand and compress the frame 102. Turning now to FIG. 2, the actuators 106 can extend from the one or more of the inflow apices 136 of the frame 102 to the one or more outflow apices 150 of the frame. The actuators may be configured to draw the inflow apices 136 and the outflow apices 150 of the frame 102 closer together, thereby axially foreshortening and radially expanding the frame 102. The actuators 106 may also be configured to move the outflow apices 150 of the frame 102 and the inflow apices 136 of the frame 102 further apart, thereby axially extending and radially compressing the frame 102.

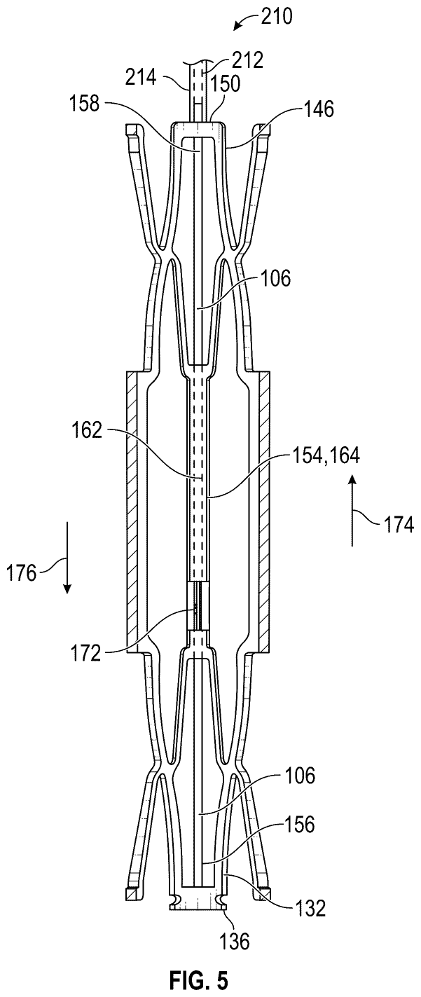

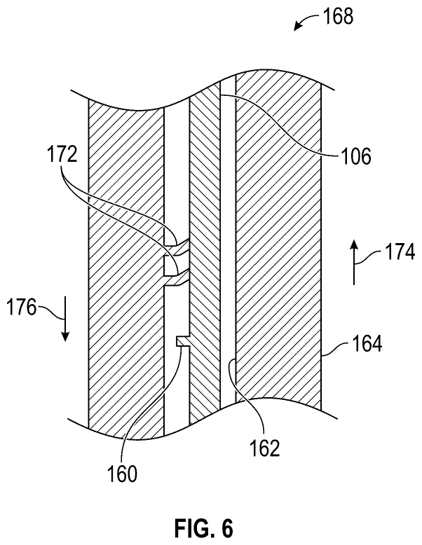

In some examples, the actuator 106 can comprise, for example, a rod or shaft. As shown in FIG. 5, the actuator 106 can comprise a fixed end portion 156 and a free end portion 158. The fixed end portion 156 can be coupled to an inflow apex, and the free end portion 158 extends toward an outflow apex 150. As shown in FIG. 6, the actuator 106 can also include a lock engagement feature 160 configured to engage with a locking element, as will be discussed in greater detail below.

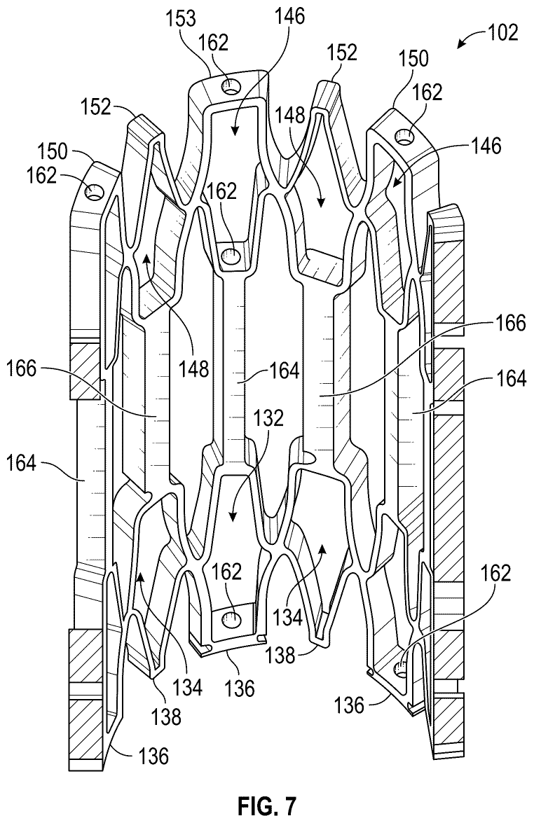

In some examples, best illustrated in FIGS. 3 and 7, the axially-extending vertical struts extending between pairs of inflow cells and outflow cells can have a bore 162 extending axially therethrough. In some examples, only some of the axially-extending vertical struts may include the bore 162 and other axially-extending vertical struts may omit the bore 162. For example, as illustrated in FIG. 7, the axially extending vertical struts comprise a first set of axially extending vertical struts 164 and a second set of axially extending struts 166. The first set of axially extending vertical struts 164 may have a vertical bore 162 extending axially therethrough, and the second set of axially extending vertical struts 166 may omit the axially-extending bore. In such examples, each of the vertical struts 164 having a vertical bore 162 extending axially therethrough can extend from an inflow cell 132 to an outflow cell 146, and each of the vertical struts 166 with no bore can extend from an inflow cell 134 to an outflow cell 148.

The bore 162 can be configured to receive an actuator, such as actuator 106 previously discussed, allowing the actuator 106 to extend from an inflow apex 136 to a corresponding outflow apex 150 of a pair of circumferentially aligned inflow and outflow cells, 132, 146, respectively, as shown in FIG. 2. In some examples, such as those shown in FIGS. 3 and 6, the bore 162 may also extend through one or both of the inflow apex 136 and the outflow apex 150 of a pair of inflow and outflow cells. This can allow the actuator 106 to extend past the inflow end 108 and/or the outflow end 110 of the frame 102. In some examples, this can allow the actuator 106 to be attached to a component of a delivery apparatus, and allow a physician to manipulate the actuator 106 during the implantation procedure.

An actuator 106 can extend through the bore 162 of each vertical strut 164. As shown in FIG. 7, the vertical struts 164 may alternate with the vertical struts 166, such that half of the vertical struts (that is, six vertical struts in the illustrated example) 154 comprise an axially extending bore 162 with an actuator 106 passing therethrough. It is to be understood, however, that in alternative examples, a greater or lesser number of the axially extending vertical struts 154 may comprise an axially extending bore 162 with an actuator 106 passing therethrough, such as 1, 2, 3, 4, 5, 7, 8, 9, 10, 11, or 12 vertical struts 154.

Each actuator 106 can be configured to radially expand the frame 102 when actuated by a corresponding actuator assembly of a delivery apparatus. As shown schematically in FIG. 5, an actuator assembly 210 of a delivery apparatus can comprise an inner actuator member 212 and an outer support tube or shaft 214 extending co-axially over the actuator member 212. The inner actuator member 212 has a distal end portion that can be releasably coupled to a proximal end portion of a corresponding actuator 106 of the prosthetic valve. The support tube 214 has a distal end portion that can abut an outflow apex 150 of the frame 102. The actuator member 212 and the support tube 214 can be moved axially relative to each other to transfer distally and proximally directed forces to the actuator 106 and the frame 102 to radially expand the frame 102.

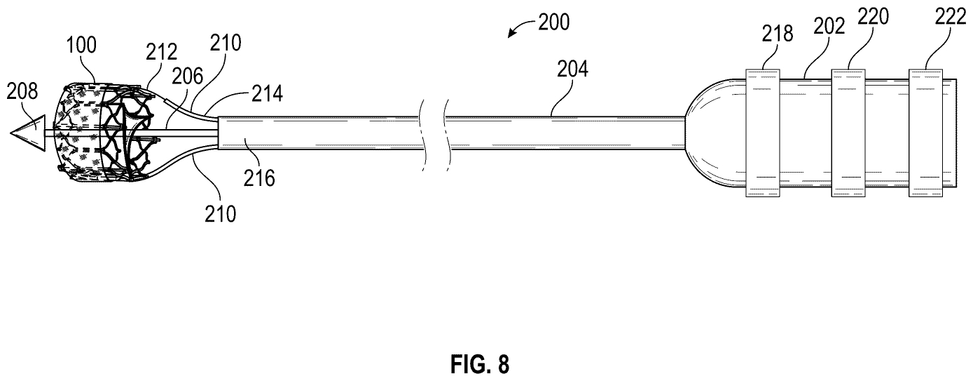

For example, moving the actuator member 212 proximally (in the direction of arrow 174) while moving the support tube 214 distally (in the direction of arrow 176) (or maintaining the support tube 214 against the apex 150 without moving the support tube) is effective to pull the actuator 106 proximally. Proximal movement of the actuator 106 is effective to move the inflow end 108 of the frame towards the outflow end 110 of the frame, thereby radially expanding the frame 102 (and the prosthetic valve 100) from the radially compressed state of FIG. 5 to the radially expanded state of FIGS. 1 and 2. Conversely, moving the actuator member 212 distally while maintaining the support tube 214 against the apex 150 is effective to push the actuator distally. Distal movement of the actuator 106 is effective to move the inflow end 108 of the frame away from the outflow end 110 of the frame, thereby radially compressing the frame 102 (and the prosthetic valve 100) from the radially expanded state to the radially compressed state. Further details regarding the operation of a delivery apparatus to control radial expansion and compression of the prosthetic valve are described below in connection with FIG. 8.

The frame 102 optionally may include a locking mechanism, such as locking mechanism 168, as illustrated in FIGS. 5-6, to assist in retaining the frame 102 in a radially expanded state. Referring now to FIG. 6, the locking mechanism 168 can include one or more retention elements 172 (which can also be referred to as “retention tabs” or “tongues” or “locking elements”) located within a bore 162 of a vertical strut 164 and configured to engage an actuator 106 located within the bore 162. Although two retention elements 172 are depicted in the illustrated example, a locking mechanism can have fewer (for example, 1) or more (for example, 3-15) than two retention elements. As shown in FIG. 5, the retention elements 172 can be located within the section of the bore 162 along the strut 164 between an inflow cell 132 and an outflow cell 146. In lieu of or in addition to the retention elements 172 located within a bore 162 of a strut, one or more retention elements 172 can be located within a bore 162 within an inflow apex 136 and/or a bore 162 of an outflow apex 150. Moreover, more than one of the bores 162 can include retention elements 172. For example, each bore 162 of a vertical strut 164 containing an actuator 106 can have one or more retention elements 172.

With continued reference to FIG. 5, when the frame 102 of the prosthetic heart valve 100 is in a radially compressed configuration (for example, a delivery configuration), a lock engagement feature 160 of the actuator 106 is spaced distally from the retention elements 172. When the frame 102 of the prosthetic heart valve 100 radially expands, the lock engagement feature 160 of the actuator 106 moves proximally toward the retention elements 172 (that is, in the direction depicted by the arrow 174). When the frame 102 of the prosthetic heart valve 100 radially compresses, the lock engagement feature 160 of the actuator 106 moves distally away from the retention elements 172 (that is, in the direction depicted by the arrow 176).

The frame 102 can be configured to freely move between various radially expanded/compressed configurations so long as the lock engagement feature 160 is disengaged from the retention elements 172. When the frame 102 is radially expanded to a desired operational diameter, the lock engagement feature 160 of the actuator 106 can engage a retention element 172. This configuration can be referred to as a locked configuration. In the locked configuration, the locking mechanism 168 prevents the actuator 106 from moving in the distal direction (arrow 176) relative to the locking mechanism 168 and therefore resists radial compression of the frame.

In alternative examples, the actuators 106 can be rotatable actuators that are configured to produce radial expansion and compression of the frame upon rotation of the actuators (similar to jack screws or screw actuators). For example, each actuator 106 can have external threads located along a portion of the length of the actuator and each bore 162 (such as a section of the bore 162 within a vertical strut 164) can have internal threads that engage the threads of an actuator 106. Thus, as the actuators 106 are rotated in a first direction, the actuators can move proximally to radially expand the frame. Conversely, as the actuators 106 are rotated in a second direction, the actuators can move distally to radially compress the frame. The engagement of the external threads and the internal threads can maintain the frame 102 in a radially expanded state without the use of any additional locking mechanism (for example, locking mechanism 168).

Further details regarding prosthetic heart valves, including locking mechanisms such as locking mechanism 168 and the ways in which locking mechanisms can be incorporated in prosthetic heart valve frames such as frame 102, actuators for radially expanding and compressing prosthetic valves, various frame constructions and methods for assembling prosthetic valves can be found in U.S. Application Nos. 63/085,947, filed Sep. 30, 2020, 63/179,766, filed Apr. 26, 2021, 63/194,285, filed May 28, 2021, and PCT Application No. PCT/US2021/040789, filed Jul. 8, 2021, which are incorporated by reference herein.

The frame 102 can be made of any of various suitable plastically-expandable materials (e.g., stainless steel, etc.) or self-expanding materials (e.g., Nitinol) as known in the art. When constructed of a plastically-expandable material, the frame 102 (and thus the valve 102) can be crimped to a radially compressed state on a delivery catheter and then expanded inside a patient by an inflatable balloon or equivalent expansion mechanism. When constructed of a self-expandable material, the frame 102 (and thus the valve 102) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

When the frame 102 is constructed from a plastically-expandable material, the expansion force required to radially expand the frame is provided by the actuators 106. In some examples, the struts 124 of the frame can be sufficiently rigid to maintain the frame 102 in the radially expanded state against a surrounding native annulus without the use of any locking mechanism 168.

When the frame 102 is constructed from a shape-memory material (for example, Nitinol), the frame 102 can be configured to self-expand from a radially compressed state to at least a partially radially expanded state or to a fully radially expanded state. In such cases, the actuators 106 can be used to assist in radially expanding the frame in cooperation with the inherent resiliency of the shape-memory material that urges the frame toward the radially expanded state. For example, the frame 102 can be self-expandable from a radially compressed state to a partially radially expanded state. After the frame reaches the partially radially expanded state, the actuators 106 can be used to further expand the frame 102 from the partially radially expanded state to a fully radially expanded state. In some examples, the frame 102 can be self-expandable from a radially compressed state to a fully radially expanded state in which the prosthetic valve 100 can contact the tissue of the native annulus. After the frame reaches the fully radially expanded state, the actuators 106 can be used to overexpand the frame and dilate the native annulus in which the prosthetic valve is implanted. One or more locking mechanisms 168, as described above, can be used to retain the frame in the overexpand state against the forces of the surrounding annulus.

Returning to FIG. 1, the valvular structure 104 of the prosthetic heart valve 100 can be coupled to the frame 102 (for example, directly and/or indirectly via other components such a sealing skirt). The valvular structure 104 is configured to allow blood flow through the prosthetic heart valve 100 from the first end 108 (that is, inflow end 108) to the second end 110 (that is, outflow end 110) in an antegrade direction and to block blood from flowing through the prosthetic heart valve 100 from the second end 110 to the first end 108 in a retrograde direction. The valvular structure can include various components including a leaflet assembly comprising two or more leaflets 178. For example, the valvular structure 104 in the illustrated example comprises a leaflet assembly having three leaflets 178. It is to be understood, however, that in some examples, the valvular structure 104 could comprise a different number of leaflets.

The leaflets 178 of the prosthetic heart valve 100 can be made of a flexible material. For example, the leaflets 178 can be made from in whole or part, biological material, bio-compatible synthetic materials, or other such materials. Suitable biological material can include, for example, bovine pericardium, equine pericardium, porcine pericardium, and/or pericardium from other sources.

The leaflets 178 can be arranged to form commissures 180. The commissures 180 can, for example, be mounted to the frame adjacent outflow apices, such as the outflow apices 152 of the outflow cells 148, as illustrated in FIG. 1. For example, each leaflet 178 can have two commissure tabs 182 on opposite sides of the leaflet 178. Each commissure tab 182 can be paired with an adjacent commissure tab 182 of an adjacent leaflet to form a respective commissure 180. Each pair of commissure tabs 182 can be coupled to a corresponding outflow cell 148, such as by sutures 184 that extend around the struts 124 forming the cell 148 and through the commissure tabs 182. Thus, a frame according to the examples disclosed herein, such as frame 102 can mount the leaflets 178 of the valvular structure 104 without the need for additional commissure windows or posts. Each commissure 180 can include one or more reinforcing members, such as fabric reinforcing members, that are sutured to the commissure tabs 182 and/or the struts 124 to reinforce the connection between the commissure tabs 182 and the struts 124.

The inflow or cusp edge portions of the leaflets 178 can be coupled to the frame 102 via various techniques and/or mechanisms. For example, the cusp edge portions of the leaflets 178 can be sutured directly to selected struts 124 of inflow cells 132 and/or cells 134. Alternatively, the cusp edge portions of the leaflets 178 can be sutured to an inner skirt (for example, a fabric skirt, not shown), which in turn can be sutured to selected struts 124 of inflow cells 132 and/or cells 134.

With continued reference to FIG. 1, the valvular structure 104 can further include an outer skirt or sealing member 186 disposed around the exterior of the frame 102. The outer skirt can be made of any suitable biocompatible and flexible material, including materials suitable for leaflets 178, or synthetic material, such as any suitable biocompatible fabric (for example, polyethylene terephthalate (PET) fabric). The outer skirt 186 can be attached to the frame 102 by means of sutures, fabric, adhesive and/or other means for mounting, and in certain examples can be attached to selected struts 124 forming the inflow cells 118 of the frame and/or vertical struts 154. The skirt 186 can be configured to improve the seal between the prosthetic heart valve 100 and the native heart valve in which the prosthetic heart valve has been implanted.

Further details regarding prosthetic heart valves, including the valvular structure 104 and manner in which the valvular structure 104 can be coupled to the frame 102 of the prosthetic heart valve 100, can be found in U.S. Pat. Nos. 6,730,118, 7,393,360, 7,510,575, 7,993,394, 8,652,202, and 9,393,110, U.S. Publication No. 2018/0325665, and U.S. Application No. 63/138,890, filed Jan. 19, 2021, which are incorporated by reference herein.

Prosthetic heart valves discussed above, such as prosthetic heart valve 100, can be configured for use with a delivery apparatus, such as delivery apparatus 200, illustrated in FIG. 8. The delivery apparatus 200 is configured to releasably attach to one or more components of the prosthetic heart valve 100, to advance the prosthetic heart valve 100 in a compressed configuration through the vasculature of the patient to a desired implantation site, and to expand the prosthetic heart valve 100 from the compressed configuration to an expanded configuration.

Turning now to FIG. 8, the delivery apparatus 200 in the illustrated example generally includes a handle 202, a first elongated shaft 204 (which comprises an outer shaft in the illustrated example) extending distally from the handle 202, at least one actuator assembly 210 extending distally from the handle through the outer shaft 204, and a second elongated shaft 206 (which comprises an inner shaft in the illustrated example) extending distally from the handle through the outer shaft 204. The at least one actuator assembly 210 can be configured to radially expand and/or radially collapse the prosthetic valve 100 when actuated. A nosecone 208 can be mounted to the distal end of the second shaft 206. The second shaft 206 and the nosecone 208 can define a guidewire lumen sized for receiving a guidewire so that the delivery apparatus can be advanced over a guidewire previously inserted into a patient's body.

In some examples, a distal end portion 216 of the shaft 204 can be sized to house the prosthetic valve in its radially compressed, delivery state during delivery of the prosthetic valve through the patient's vasculature. In this manner, the distal end portion 216 functions as a delivery sheath or capsule for housing the prosthetic valve during delivery.

As noted above, each actuator assembly 210 can include an actuator member 212 extending through a support tube or support member 214. In alternative examples, the delivery apparatus actuator members 212 and the support members 214 need not be co-axial with respect to each and instead can extend side-by-side through the outer shaft 204. Each actuator member 212 can have a distal end portion releasably coupled to a component of the prosthetic heart valve 100, such as to a corresponding actuator 106. Although the illustrated example shows two actuator assemblies 210 for purposes of illustration, it should be understood that one actuator assembly 210 can be provided for each actuator 106 on the prosthetic valve. For example, six actuator assemblies 210 can be provided for a prosthetic valve having six actuators 106. In some examples, a greater or fewer number of actuator assemblies can be present

The delivery apparatus actuators 212 and/or the support members 214 can be configured to radially expand the prosthetic heart valve 100 by exerting an axial force on the actuators 106, thereby drawing the inflow end 108 and the outflow end 110 of the prosthetic heart valve 100 closer together, axially foreshortening and radially expanding the prosthetic heart valve 100, as previously discussed. As one example, a physician can move the delivery apparatus actuators 212 proximally to provide a proximally directed force to the actuators 106 of the prosthetic heart valve 100, while simultaneously gripping, holding, and/or pushing the handle 202 to provide the countervailing distally directed force to the proximal or outflow end 110 of the prosthetic valve 100 via the support members 214.

The delivery apparatus actuators 212 can comprise a suture, string, cord, wire, cable, shaft, rod, or other similar device that can transmit a pulling force from the handle 202 to the prosthetic valve when actuated by a physician. The support members 214 can comprise a relatively more rigid component, such a tube that can abut the proximal end 110 (the outflow end 110 in the illustrated example) of the prosthetic heart valve 100 and resist proximal movement of the prosthetic valve relative to the outer shaft 204 when a proximal pulling force is applied to the actuators 106.

The distal end portions of the delivery apparatus actuators 212 of the delivery apparatus 200 can be releasably coupled to corresponding actuators 106 of the prosthetic heart valve 100 in various ways. For example, the distal end portion of each delivery apparatus actuator 212 can comprise external threads and the proximal end portion 158 of each actuator 106 can comprise internal threads (or vice versa), thereby enabling each delivery apparatus actuator 212 and corresponding actuator 106 to be threadably coupled together.

The delivery apparatus actuators 212 of the delivery apparatus 200 can be sized to have approximately the same diameter as actuators 106, and configured to axially move within the bores 162 in the vertical struts 154. In such examples, the actuators 212 may extend at least partially through the bores while the prosthetic heart valve 100 is in a radially compressed (or axially extended) configuration. The delivery apparatus actuators 212 can then axially move towards the proximal end 110 of the prosthetic heart valve 100 and draw the actuators 106 in the same direction, thereby axially foreshortening and radially expanding the prosthetic heart valve 100. When the prosthetic valve 100 is fully radially expanded, the distal end portions of the delivery apparatus actuators 212 can be positioned proximally of the outflow apices 150 of the frame for releasing the delivery apparatus actuators 212 from the actuators 106 of the prosthetic valve.

Various other types of releasable connections between the delivery apparatus actuators 212 and the prosthetic heart valve actuators 106 can be used, such as those disclosed in in U.S. Application No. 63/194,285, which is incorporated by reference herein.

The handle 202 of the delivery apparatus 200 can include one or more control mechanisms (for example, knobs or other actuating mechanisms) for controlling different components of the delivery apparatus 200 in order to expand and/or deploy the prosthetic valve 100. For example, in the illustrated example the handle 202 comprises first, second, and third knobs 218, 220, 222, respectively.

The second knob 220 can be a rotatable knob configured to produce radial expansion and/or contraction of the prosthetic valve 100. For example, rotation of the second knob 220 can move the actuator members 212 and the support tubes 214 axially relative to one another. Rotation of the second knob 220 in a first direction (for example, clockwise) can radially expand the prosthetic valve 100 and rotation of the second knob 220 in a second direction (for example, counter-clockwise) can radially collapse the prosthetic valve 100. In some examples, the second knob 220 can be actuated by sliding or moving the knob 220 axially, such as pulling and/or pushing the knob.

The third knob 222 can be operatively connected to a proximal end portion of each actuator member 212. The fourth knob 222 can be configured to rotate each actuator member 212, upon rotation of the knob, to unscrew each actuator member 212 from the proximal portion of a respective actuator 106 when the prosthetic valve is fully expanded at the desired implantation location.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire and/or an introducer sheath previously inserted into a patient's vasculature. Moreover, the disclosed delivery approaches are not intended to be limited. Any of the prosthetic valves disclosed herein can be implanted using any of various delivery procedures and delivery devices known in the art.

Advantageously, prosthetic heart valves according to the examples previously discussed can include a greater number of inflow and outflow cells, reducing empty spaces between adjacent inflow cells and between adjacent outflow cells, and offering improved support and protection to the valvular structure of the prosthetic heart valve. The additional cells may also provide improved support, and additional attachment points for the leaflets of the valvular structure. Additionally, prosthetic heart valves using frames according to the examples previously discussed may have improved resistance to crushing or compression by the native vasculature of the patient, and may be more stable within the patient once implanted.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve assembly, comprising a radially expandable annular frame comprising a plurality of interconnected struts that form an inflow row of cells defining an inflow end of the frame, a plurality of interconnected struts that form an outflow row of cells defining an outflow end of the frame and circumferentially aligned with corresponding cells of the inflow row, a plurality of axially extending struts interconnecting pairs of corresponding inflow and outflow cells, and at least one actuation member extending through one of the axially extending struts and an inflow cell and an outflow cell of a selected pair of inflow and outflow cells from the pairs of corresponding inflow and outflow cells, wherein the actuation member is configured to radially expand and compress the frame; and a valvular structure disposed within the frame and configured to regulate the flow of blood through the frame from the inflow end to the outflow end.

Example 2. The prosthetic heart valve assembly of any example herein, particularly example 1, wherein the actuation member extends from an inflow apex of the inflow cell to an outflow apex of the outflow cell of the selected pair of inflow and outflow cells.

Example 3. The prosthetic heart valve assembly of example herein, particularly examples 1-2, wherein the inflow row comprises six or more cells and the outflow row comprises six or more cells.

Example 4. The prosthetic heart valve assembly of example herein, particularly example 3, wherein the inflow row comprises exactly twelve cells and the outflow row comprises exactly twelve cells.

Example 5. The prosthetic heart valve assembly of any of example herein, particularly examples 1-4, wherein the actuation member extends through an axially extending bore of the corresponding axially extending strut.

Example 6. The prosthetic heart valve assembly of any example herein, particularly examples 1-5, wherein the least one actuation member comprises a plurality of actuation members extending through corresponding axially extending struts and corresponding pairs of inflow and outflow cells.

Example 7. The prosthetic heart valve assembly of any example herein, particularly example 6, wherein the pairs of corresponding inflow and outflow cells comprise a first set of pairs of inflow and outflow cells and a second set of pairs of inflow and outflow cells, wherein each pair of inflow and outflow cells of the first set is located circumferentially between two pairs of inflow and outflow cells of the second set, wherein each of the actuation members extends through a pair of inflow and outflow cells of the first set.

Example 8. The prosthetic heart valve assembly of any example herein, particularly example 7, wherein the outflow cells of the second set define first apices and the outflow cells of the first set define second apices, wherein the distance from the first apices to an axial mid-section of the frame is less than the distance from the second apices to the axial mid-section of the frame.

Example 9. The prosthetic heart valve assembly of any example herein, particularly example 8, wherein the inflow cells of the second set define third apices and the inflow cells of the first set define fourth apices, wherein the distance from the third apices to an axial mid-section of the frame is less than the distance from the fourth apices to the axial mid-section of the frame.

Example 10. The prosthetic heart valve of any example herein, particularly examples 7-9, wherein the valvular structure comprises a plurality of leaflets defining a plurality of commissures, wherein each commissure is coupled to an outflow cell of the second set.

Example 11. The prosthetic heart valve assembly of any example herein, particularly examples 1-10, wherein the inflow and outflow cells are diamond shaped.

Example 12. The prosthetic heart valve assembly of any example herein, particularly examples 1-11, wherein the frame comprises a circumferentially extending row of hexagonal cells, each hexagonal cell defined by two adjacent axially extending struts, a first strut of an inflow cell, a second strut of another, adjacent inflow cell, a third strut of an outflow cell, and a fourth strut of another, adjacent outflow cell.

Example 13. The prosthetic heart valve assembly of any example herein, particularly example 1-12, wherein the frame further comprises a locking mechanism configured to prevent radial compression of the frame when engaged.

Example 14. The prosthetic heart valve assembly of any example herein, particularly examples 1-13, further comprising an outer skirt disposed around an outer surface of the frame, wherein the outer skirt comprises an inflow edge sutured to struts forming the inflow cells.

Example 15. A radially expandable frame comprising a row of inflow cells circumferentially arranged in an annular configuration at an inflow end portion of the frame and a row of outflow cells circumferentially arranged in an annular configuration at an outflow end portion of the frame wherein each inflow cell is circumferentially aligned with an outflow cells to define pairs of circumferentially aligned inflow and outflow cells; a row of intermediate cells circumferentially arranged in an annular configuration and disposed between the row of inflow cells and the row of outflow cells; a plurality of axially extending vertical struts connecting pairs of inflow and outflow cells and forming sides of the intermediate cells; and at least one actuation device extending from an inflow cell to an outflow cell of a selected pair of inflow and outflow cells wherein the actuation device is configured to radially expand frame from a radially compressed state to a radially expanded state.

Example 16. The radially expandable frame of any example herein, particularly example 15, wherein the actuation device extends from an apex of the inflow cell and an apex of the outflow cell.

Example 17. The radially expandable frame of any example herein, particularly examples 15-16, wherein the actuation device passes through an axially-extending bore through a vertical strut.

Example 18. The radially expandable frame of any example herein, particularly examples 15-17, wherein the radially expandable frame comprises six or more pairs of circumferentially aligned inflow and outflow cells.

Example 19. The radially expandable frame of any example herein, particularly examples 15-18, wherein the radially expandable frame comprises exactly twelve pairs of circumferentially aligned inflow and outflow cells.

Example 20. The radially expandable frame of any example herein, particularly examples 15-19, wherein the at least one actuator comprises a plurality of actuators extending from a plurality of inflow cells to a plurality of outflow cells.

Example 21. The radially expandable frame of any example herein, particularly example 20, wherein the plurality of actuators each extend from an apex of an inflow cell to an apex of an outflow cells.

Example 22. The radially expandable frame of any example herein, particularly examples 15-21, wherein the plurality of inflow cells and the plurality of outflow cells form a plurality of selected pairs of inflow and outflow cells.

Example 23. The radially expandable frame of any example herein, particularly examples 15-22, wherein pairs of corresponding inflow and outflow cells comprise a first set of cell pairs and a second set of cell pairs, wherein each cell pair of the first set of cell pairs is located circumferentially between two cell pairs of the second set of cell pairs.

Example 24. The radially expandable frame of any example herein, particularly example 23, wherein each actuation device extends between a select pair of an inflow cell and an outflow cell of the first set of cell pairs.

Example 25. The radially expandable frame of any example herein, particularly example 24 wherein each of the first set of cell pairs is connected by an actuator.

Example 26. The radially expandable frame of any example herein, particularly example 24, wherein at least one cell pair of the first set of cell pairs is not connected by an actuator.

Example 27. The radially expandable frame of any example herein, particularly examples 23-26, wherein the inflow cells of the first set of cells form a first set of inflow apices and the inflow cells of the second set of cells form a second set of inflow apices, and wherein the distance between the first set of inflow apices and an axial mid-section of the frame is greater than the distance between the second set of apices and the axial mid-section of the frame.

Example 28. The radially expandable frame of any example herein, particularly examples 23-26, wherein the outflow cells of the first set of cells form a first set of outflow apices and the outflow cells of the second set of cells form a second set of outflow apices, and wherein the distance between the first set of outflow apices and an axial mid-section of the frame is greater than the distance between the second set of apices and the axial mid-section of the frame.

Example 29. The radially expandable frame of any example herein, particularly examples 15-28, wherein the frame supports a valvular structure comprising a plurality of leaflets forming a plurality of commissures and configured to regulate the flow of blood through the frame from the inflow end to the outflow end.

Example 30. The radially expandable frame of any example herein, particularly example 29, wherein each commissure of the valvular structure is connected to an outflow cell of the radially expandable frame.

Example 31. The radially expandable frame of any example herein, particularly examples 15-30, wherein the inflow cells and the outflow cells each have a diamond shape.

Example 32. The radially expandable frame of any example herein, particularly examples 15-31, wherein the intermediate cells are hexagonal cells formed defined by two vertical struts, two adjacent inflow cells, and two adjacent outflow cells.

Example 33. The radially expandable frame of any of claims 15-32, wherein the radially expandable frame comprises a locking mechanism configured to prevent further radial expansion or radial contraction of the frame when engaged.

Example 34. A radially expandable frame, comprising a plurality of diamond-shaped outflow cells, a plurality of diamond-shaped inflow cells, a plurality of hexagonal-shaped intermediate cells disposed between the outflow cells and the inflow cells, and at least one actuator extending from an inflow cell to an outflow cell and configured to radially expand and radially contract the radially expandable frame.

Example 35. The radially expandable frame of any example herein, particularly example 34, wherein each inflow cell is circumferentially aligned with an outflow cell to form a pair of corresponding inflow and outflow cells.

Example 36. The radially expandable frame of any example herein, particularly examples 34-35, wherein the radially expandable frame comprises a plurality of axially-extending vertical struts extending from the plurality of diamond-shaped inflow cells to the plurality of diamond-shaped outflow cells.

Example 37. The radially expandable frame of any example herein, particularly example 36, wherein the number of axially-extending vertical struts is equal to the number of inflow cells and to the number of outflow cells, and each axially extending vertical strut extends from a different inflow cell to a different outflow cell.

Example 38. The radially expandable frame of any example herein, particularly examples 34-37, wherein the hexagonal intermediate cells are defined by two axially extending vertical struts, two inflow cells, and two outflow cells.

Example 39. The radially expandable frame of any example herein, particularly examples 34-38, wherein the radially expandable frame comprises more than six inflow cells, more than six outflow cells, and more than six intermediate cells.

Example 40. The radially expandable frame of any example herein, particularly examples 34-38, wherein the radially expandable frame comprises exactly twelve inflow cells, exactly twelve outflow cells, and exactly twelve intermediate cells.

Example 41. The radially expandable frame of any example herein, particularly examples 36-40, wherein the actuator extends through an axially extending bore through an axially extending vertical strut.

Example 42. The radially expandable frame of any example herein, particularly examples 34-41, wherein the at least one actuator comprises a plurality of actuators, each extending from an inflow cell to an outflow cell.

Example 43. The radially expandable frame of any example herein, particularly example 35, wherein the pairs of corresponding inflow and outflow cells comprise a first set of pairs of inflow and outflow cells and a second set of pairs of inflow and outflow cells.

Example 44. The radially expandable frame of any example herein, particularly example 43, wherein each pair of first set of pairs of inflow and outflow cells are disposed circumferentially between two pairs of the second set of pairs inflow and outflow cells and each pair of the second set of pairs of inflow and outflow cells are disposed circumferentially between two pairs of the first set of pairs of inflow and outflow cells.

Example 45. The radially expandable frame of any example herein, particularly example 44, wherein the inflow cells of the first set of cell pairs comprise a first set of inflow apices and the inflow cells of the second set of cell pairs comprise a second set of inflow apices, wherein the distance from the first set of inflow apices to an axial midpoint of the radially expandable frame is greater than the distance from the second set of inflow apices to the axial midpoint of the radially expandable frame.

Example 46. The radially expandable frame of any example herein, particularly examples 44-45, wherein the outflow cells of the first set of cell pairs comprise a first set of outflow apices and the outflow cells of the second set of cell pairs comprise a second set of outflow apices, wherein the distance from the first set of outflow apices to an axial midpoint of the radially expandable frame is greater than the distance from the second set of outflow apices to the axial midpoint of the radially expandable frame.

Example 47. The radially expandable frame of any example herein, particularly examples 44-46, wherein the actuator extends from an inflow cell of the first set of cell pairs to an outflow cell of the first set of cell pairs.

Example 48. The radially expandable frame of any example herein, particularly examples 44-47, wherein the one or more actuators comprise a plurality of actuators, and an actuator extends from each inflow cell of the first set of cell pairs to a corresponding outflow cell of the first set of cell pairs.

Example 49. The radially expandable frame of any example herein, particularly examples 43-48, wherein the radially expandable frame supports a valvular structure comprising a plurality of leaflets forming a plurality of commissures and configured to regulate the flow of blood through the frame from the inflow end to the outflow end.

Example 50. The radially expandable frame of any example herein, particularly examples 43-49, wherein each of the plurality of commissures is attached to an outflow cell of the second set of cell pairs.

Example 51. The radially expandable frame of any example herein, particularly examples 34-50, wherein the radially expandable frame comprises a locking mechanism configured to prevent further radial expansion or radial contraction of the frame when engaged.

Example 52. A radially expandable frame, comprising an inflow row of cells defining an inflow end of the radially expandable frame, an outflow row of cells defining an outflow end of the radially expandable frame, a plurality of axially extending vertical struts connecting the inflow row of cells with the outflow row of cells to define a plurality of pairs of end cells comprising an inflow cell and a corresponding outflow cell, an intermediate row of cells disposed between the inflow row of cells and the outflow row of cells and defined by two vertical struts, two cells of the inflow row of cells, and two cells of the outflow row of cells, and at least one actuator extending from the inflow cell of a pair of end cells to the corresponding outflow cell of the pair of end cells; wherein each inflow cell has an inflow apex at the inflow end of the radially expandable frame and an interior vertex located between the inflow apex and the corresponding outflow cell, and each outflow cell has an outflow apex at the outflow end of the radially expandable frame and an interior vertex located between the outflow apex and the corresponding inflow cell, such that the interior vertices of each pair of end cells face each other, wherein the cells of the intermediate row of cells are larger than the cells of the outflow row of cells and the cells of the inflow row of cells.

Example 53. The radially expandable frame of any example herein, particularly example 52, wherein the actuator extends through the inflow cell and the corresponding outflow cell of the end cell pair.

Example 54. The radially expandable frame of any example herein, particularly examples 52-53, wherein the end cells are diamond shaped.

Example 55. The radially expandable frame of any example herein, particularly examples 52-54, wherein the intermediate cells are hexagonal.

Example 56. The radially expandable frame of any example herein, particularly examples 52-55, wherein the intermediate cells are longer in the axial direction than the end cells.

Example 57. The radially expandable frame of any example herein, particularly examples 52-56, wherein the row of inflow cells comprises more than 6 cells, the row of outflow cells comprises more than 6 cells, and the row of intermediate cells comprises more than 6 cells.

Example 58. The radially expandable frame of any example herein, particularly examples 52-57, wherein the row of inflow cells comprises exactly 12 cells, the row of outflow cells comprises exactly 12 cells, and the row of intermediate cells comprises exactly 12 cells.

Example 59. The radially expandable frame of any example herein, particularly examples 52-58, wherein each cell in the row of intermediate cells comprise a first intermediate vertex defined by two inflow cells and a second intermediate vertex defined by two outflow cells, wherein the first intermediate vertices are disposed circumferentially between the interior vertices of the inflow cells and the second intermediate vertices are disposed circumferentially between the interior vertices of the outflow cells.

Example 60. The radially expandable frame of any example herein, particularly examples 52-59, wherein the one or more actuators comprises a plurality of actuators and each actuator extends from the inflow cell of a pair of end cells to the corresponding outflow cell.

Example 61. The radially expandable frame of any example herein, particularly examples 52-60, wherein the axially-extending struts comprise an axially-extending bore and each actuator passes through a bore in an axially extending strut.

Example 62. The radially expandable frame of any example herein, particularly examples 52-61, wherein the plurality of pairs of end cells comprises a first set of pairs and a second set of pairs, wherein each pair of the first set of pairs is disposed between two pairs of the second set of pairs and each pair of the second set of pairs is disposed between two pairs of the first set of pairs.

Example 63. The radially expandable frame of any example herein, particularly example 62, wherein each actuator extends from an inflow cell to a corresponding outflow cell of the first set of pairs.

Example 64. The radially expandable frame of any example herein, particularly examples 62-63, wherein an actuator extends from each inflow cell of the first set of pairs to the corresponding outflow cells.

Example 65. The radially expandable frame of any example herein, particularly examples 62-64, wherein the inflow cells of the first set of pairs comprise a first set of inflow apices and the inflow cells of the second set of pairs comprise a second set of inflow apices, and the distance from the first set of inflow apices to an axial midpoint of the frame is greater than the distance from the second set of inflow apices to the axial midpoint of the frame.

Example 66. The radially expandable frame of any example herein, particularly examples 62-65, wherein the outflow cells of the first set of pairs comprise a first set of outflow apices and the outflow cells of the second set of pairs comprise a second set of outflow apices, and the distance from the first set of outflow apices to an axial midpoint of the frame is greater than the distance from the second set of inflow apices to the axial midpoint of the frame.

Example 67. The radially expandable frame of any example herein, particularly examples 62-66, wherein the radially expandable frame supports a valvular structure comprising a plurality of leaflets forming a plurality of commissures and configured to regulate the flow of blood from the inflow end to the outflow end.

Example 68. The radially expandable frame of any example herein, particularly example 67, wherein the each of the plurality of commissures is attached to an outflow cell of the second set of pairs.

Example 69. The radially expandable frame of any example herein, particularly examples 52-68, wherein the radially expandable frame comprises a locking mechanism configured to prevent radial expansion and radial contraction of the frame when engaged.

Example 70. A radially expandable frame, comprising a plurality of frame sections comprising a plurality of circumferentially-extending interconnecting angled struts, a plurality of axially-extending vertical struts, a diamond-shaped first inflow cell defined by four interconnected angled struts and having a first inflow apex, a diamond-shaped first outflow cell defined by four interconnected angled struts and having a first outflow apex, two hexagonal-shaped intermediate cells defined by two vertical struts and four angled struts, wherein the first inflow cell of each frame section is circumferentially aligned with the first outflow cell of each frame section and the hexagonal shaped intermediate cells of each frame section are axially aligned with each other and wherein the axially-extending vertical struts extend from the first inflow cell of each frame section to the first outflow cell of each frame section; a plurality of second inflow cells each having a second inflow apex and disposed between the first inflow cells of two frame sections; a plurality of second outflow cells, each having a second outflow apex and disposed between the first outflow cells of two frame sections; and at least one actuation member extending from a first inflow cell of a frame section to the corresponding outflow frame cell of that frame section.

Example 71. The radially expandable frame of any example herein, particularly example 70, wherein the radially expandable frame comprises at least three frame sections, at least three second inflow cells, and at least three second outflow cells.

Example 72. The radially expandable frame of any example herein, particularly examples 70-71, wherein the radially expandable frame comprises exactly six frame sections, exactly six second inflow cells, and exactly six second outflow cells.

Example 73. The radially expandable frame of any example herein, particularly examples 70-72 wherein the distance from the first inflow apices to an axial midpoint of the radially expandable frame is greater than the distance from the second inflow apices to the axial midpoint of the radially expandable frame.

Example 74. The radially expandable frame of any example herein, particularly examples 70-73, wherein the at least one actuation member comprises a plurality of actuation members.

Example 75. The radially expandable frame of any example herein, particularly examples 70-74, wherein each actuator extends from the first inflow apex of a cell section to the corresponding first outflow apex of the cell section and passes through an axial bore through an axially-extending vertical strut.

Example 76. The radially expandable frame of any example herein, particularly examples 70-75, wherein each pair of first inflow cells and first outflow cells have an actuator extending therebetween.

Example 77. The radially expandable frame of any example herein, particularly examples 70-76, wherein the radially expandable frame supports a valvular structure comprising a plurality of leaflets forming a plurality of commissures and configured to regulate the flow of blood from the inflow end to the outflow end.

Example 78. The radially expandable frame of any example herein, particularly example 77, wherein each commissure is attached to a second outflow cell.

Example 79. The radially expandable frame of any example herein, particularly examples 70-78, wherein the radially expandable frame comprises a locking mechanism configured to prevent the radial expansion and radial contraction of the radially expandable frame when engaged.

Example 80. A prosthetic valve heart comprising a radially expandable frame of any of examples 15-79 and a valvular structure positioned within the frame.

Example 81. A method comprising sterilizing the commissure assembly, the radially expandable frame, the leaflets, or the prosthetic heart valve of any preceding claim.

Example 82. An expandable frame, leaflets, or prosthetic heart valve of any preceding claim, wherein the expandable frame, leaflets, or prosthetic heart valve is sterilized.

In view of the many possible examples to which the principles of the disclosed invention may be applied, it should be recognized that the illustrated examples are only preferred examples of the invention and should not be taken as limiting the scope of the invention. Rather, the scope of the invention is defined by the following claims. We therefore claim as our invention all that comes within the scope and spirit of these claims.

1. A prosthetic heart valve assembly, comprising:
a radially expandable annular frame comprising:
a plurality of interconnected struts that form a row of inflow cells defining an inflow end of the frame;
a plurality of interconnected struts that form a row of outflow cells defining an outflow end of the frame and circumferentially aligned with corresponding cells of the inflow row of cells to form a plurality of pairs of inflow and outflow cells,
a plurality of axially extending struts, each axially extending strut interconnecting an outflow cell and an inflow cell of a respective pair;
an actuation member extending through a first axially extending strut of the plurality of axially extending struts and an inflow cell and an outflow cell of a selected pair of inflow and outflow cells from the plurality of pairs of inflow and outflow cells, wherein the actuation member is configured to radially expand and compress the frame; and
a valvular structure disposed within the frame and configured to regulate blood flow through the frame from the inflow end to the outflow end.

2. The prosthetic heart valve assembly of claim 1, wherein the actuation member extends from an inflow apex of a first inflow cell to an outflow apex of a first outflow cell of the selected pair of inflow and outflow cells.

3. The prosthetic heart valve assembly of claim 1, wherein the inflow row of cells comprises six or more cells and the outflow row of cells comprises six or more cells.

4. The prosthetic heart valve assembly of claim 3, wherein the inflow row of cells comprises exactly twelve cells and the outflow row of cells comprises exactly twelve cells.

5. The prosthetic heart valve assembly of claim 1, wherein the actuation member extends through an axially extending bore in the first axially extending strut.

6. The prosthetic heart valve assembly of claim 1, further comprising one or more additional actuation members, wherein each additional actuation member extends through an axially extending strut and a corresponding pair of inflow and outflow cells.

7. The prosthetic heart valve assembly of claim 6, wherein the plurality of pairs of inflow cells and outflow cells comprise a first set of pairs and a second set of pairs, wherein each pair of inflow cells and outflow cells of the first set of pairs is located circumferentially between two pairs of inflow and outflow cells of the second set of pairs.

8. The prosthetic heart valve assembly of claim 7, wherein the outflow cells of the second set of pairs define first apices and the outflow cells of the first set of pairs define second apices, wherein a first distance from the first apices to an axial midpoint of the frame is less than a second distance from the second apices to the axial midpoint of the frame.

9. The prosthetic heart valve assembly of claim 8, wherein the inflow cells of the second set of pairs define third apices and the inflow cells of the first set of pairs define fourth apices, wherein a third distance from the third apices to an axial midpoint of the frame is less than a fourth distance from the fourth apices to the axial midpoint of the frame.

10. The prosthetic heart valve assembly of claim 1, wherein the inflow row of cells and outflow row of cells comprise diamond shaped cells.

11. The prosthetic heart valve assembly of claim 1, wherein the frame comprises a circumferentially extending row of hexagonal cells, each hexagonal cell defined by two adjacent axially extending struts, a first strut of an inflow cell, a second strut of another, adjacent inflow cell, a third strut of an outflow cell, and a fourth strut of another, adjacent outflow cell.

12. A radially expandable frame comprising:
a plurality of inflow cells circumferentially arranged in an annular configuration at an inflow end portion of the frame;
a plurality of outflow cells circumferentially arranged in an annular configuration at an outflow end portion of the frame;
wherein each inflow cell is circumferentially aligned with an outflow cell to define pairs of circumferentially aligned inflow and outflow cells;
a plurality of intermediate cells circumferentially arranged in an annular configuration and disposed between the inflow cells and the outflow cells;
a plurality of axially extending struts connecting pairs of inflow and outflow cells and forming sides of the intermediate cells; and
at least one actuation device extending from an inflow cell to an outflow cell of a selected pair of inflow and outflow cells; and
wherein the actuation device is configured to radially expand frame from a radially compressed state to a radially expanded state.

13. The radially expandable frame of claim 12, wherein the radially expandable frame comprises six or more pairs of circumferentially aligned inflow and outflow cells.

14. The radially expandable frame of claim 12, wherein the at least one actuator comprises a plurality of actuators, each actuator of the plurality of actuators extending from an inflow cell to an outflow cell of a respective pair of inflow and outflow cells.

15. The radially expandable frame of claim 12, wherein pairs of corresponding inflow cells and outflow cells comprise a first set of cell pairs and a second set of cell pairs, wherein each cell pair of the first set of cell pairs is located circumferentially between two cell pairs of the second set of cell pairs.

16. The radially expandable frame of claim 15, wherein the inflow cells of the first set of cell pairs form a first set of inflow apices and the inflow cells of the second set of cell pairs form a second set of inflow apices, and wherein a first distance between the first set of inflow apices and an axial midpoint of the frame is greater than a second distance between the second set of inflow apices and the axial midpoint of the frame.

17. The radially expandable frame of claim 15, wherein the outflow cells of the first set of cell pairs form a first set of outflow apices and the outflow cells of the second set of cell pairs form a second set of outflow apices, and wherein a third distance between the first set of outflow apices and an axial midpoint of the frame is greater than a fourth distance between the second set of outflow apices and the axial midpoint of the frame.

18. The radially expandable frame of claim 12, wherein the inflow cells and the outflow cells each have a diamond shape.

19. The radially expandable frame of claim 12, wherein the intermediate cells are hexagonal cells formed defined by two axially extending struts, two adjacent inflow cells, and two adjacent outflow cells.

20. A radially expandable frame, comprising:
a row of inflow cells defining an inflow end of the radially expandable frame;
a row of outflow cells defining an outflow end of the radially expandable frame;
a plurality of axially extending struts connecting the row of inflow cells with the row of outflow cells to define a plurality of pairs of cells comprising an inflow cell and a corresponding outflow cell;
a row of intermediate cells disposed between the row of inflow cells and the row of outflow cells and defined by two axially extending struts, two cells of the row of inflow cells, and two cells of the row of outflow cells; and
at least one actuator extending from the inflow cell of a pair of end cells to the corresponding outflow cell of the pair of end cells;
wherein each inflow cell has an inflow apex at the inflow end of the radially expandable frame and a first interior vertex located between the inflow apex and the corresponding outflow cell, and each outflow cell has an outflow apex at the outflow end of the radially expandable frame and a second interior vertex located between the outflow apex and a corresponding inflow cell, such that the first interior vertex and the second interior vertex face each other; and
wherein the cells of the intermediate row of cells are larger than the cells of the outflow row of cells and the cells of the inflow row of cells.
